(19) United States
(12) Patent Application Publication
(10) Pub. No.: US 2011/0213331 A1
(43) Pub. Date: Sep. 1, 2011

(54) APPARATUS FOR CONTROLLING DRUG INFUSION AND METHOD USING THE SAME

(75) Inventors: Jin-Sung Kim, Anyang-si (KR); Chang-Woo Song, Anyang-si (KR); Sang-Seop Han, Anyang-si (KR)

(73) Assignee: Korea Research Institute of Chemical Technology

(21) Appl. No.: 12/599,376

(22) PCT Filed: Mar. 10, 2008
(86) PCT No.: PCT/KR08/01334

§ 371 Date: Nov. 16, 2010

(30) Foreign Application Priority Data
May 15, 2007 (KR) 10-2007-0047144

(51) Int. Cl. A61M 5/168

(52) U.S. Cl. 604/503; 604/66

(57) ABSTRACT

A drug infusion control apparatus and method that can improve precision of experiments and thus improve reliability of data obtained from experiments on experimental animals by automatically calculating an infusion amount of a drug in accordance with weights of the experimental animals included in a variety of experimental groups and infusing the calculated amount of the drug into the experimental animals are provided. The drug infusion control method includes calculating and storing a drug infusion amount using weight data of experimental animals according to a preset formula, loading data on the drug infusion amount data that is stored, determining an operation time of an opening/closing valve, receiving an operation signal from an infusion switch, opening the opening/closing valve for a predetermined time, and repeatedly infusing a drug in response to the loaded data by a preset value.

### TECHNICAL FIELD

The present invention relates to an apparatus for controlling drug infusion and a method for controlling the drug infusion using the apparatus. More particularly, the present invention relates to a drug infusion controlling apparatus that can be used for performing an experiment for infusing a drug to experimental animals and observing drug infusion results, and can improve the accuracy and speed of experiments on animals by automatically calculating an infusion amount of the drug in accordance with weight of the experimental animals and infusing the calculated amount of the drug to the experimental animals in a process for repeatedly infusing the drug and a drug infusion control method using the apparatus.

### BACKGROUND ART

Generally, in a process for developing medical supplies, agricultural chemicals, and the like, a new potential substance is infused into experimental animals such as rodents (e.g., mice and rats) and primates (e.g., monkeys) to perform a pre-clinical demonstration (or non-clinical demonstration) before a clinical demonstration is performed on human bodies. The new potential substance may be repeatedly infused into the experimental animals using a drug infusion device for oral administration and inhalation administration, and a drug supplying device.

In order to perform a pre-clinical demonstration, there is a need to calculate and vary an infusing amount of the drug in accordance with weight of the experimental animals included in a variety of experiment groups. That is, the infusing amount by weight of the experimental animals fluctuates in accordance with experimental conditions (e.g., groups (a low dose group and a high dose group) in which the animals are included), a food absorption capability of the animals, sensitivity to the environment, and the like. Therefore, if an infusion amount of the drug into the animals is not calculated considering the weight and the repeated drug infusion, and if automation of the drug infusion is not realized, the precision of the experiments is deteriorated and the experiments cannot be quickly performed.

### DISCLOSURE

### Technical Problem

Therefore, the present invention has been made in an effort to solve the above-described limitations. It is an object of the present invention to provide a drug infusion control apparatus that can improve precision of experiments and thus improve reliability of data obtained from the experiments on experimental animals by automatically calculating an infusion amount of a drug in accordance with weight of the experimental animals included in a variety of experimental groups and infusing the calculated amount of the drug into the experimental animals, and a drug infusion control method using the apparatus.

### Technical Solution

In order to achieve the object, the present invention provides a drug infusion control apparatus including a drug supplying unit including an opening/closing valve, a drug infusion unit for infusing a drug supplied from the drug supplying unit into experimental animals, and an infusion amount controller for controlling the opening/closing valve of the drug supplying unit in accordance with a signal from an infusion switch of the drug infusion unit, wherein the infusion amount controller includes an infusion amount storage database that stores data on weight of the experimental animals and an infusion amount, and a controller that controls the opening/closing valve to infuse the drug infusion amount that is calculated based on the weight data of the experimental animals stored in the infusion amount storage database into the experimental animals in accordance with a signal from the infusion switch.

The drug supplying unit may further include a drug storing unit for storing the drug that will be infused into the experimental animals, and a dispenser or pump for dispensing the drug stored in the drug storing unit.

The drug infusion unit may further include an infusion member for infusing the drug from the drug supplying unit into the experimental animals, an image pickup unit for capturing an image of an infusion state of the drug, and a display unit for outputting the image that is captured by the image pickup unit to a display unit.

The infusion amount controller may further include an image storage database for storing the image that is captured by the image pickup unit of the drug infusion unit.

The infusion amount controller may further include a backup storage database for storing the infusion amount data and the image data.

### Advantageous Effects

According to the present invention, a drug infusion amount is automatically calculated and stored by a controller and an infusion unit in accordance with weight of experimental animals of each of experimental groups. Therefore, precision of experiments can be improved and the experiments can be quickly performed. Thus, reliability of experimental data on the animals can be improved.

### DESCRIPTION OF DRAWINGS

FIG. 1 is a schematic diagram of a drug infusion control apparatus according to an exemplary embodiment of the present invention.

FIG. 2 is a diagram illustrating an example of an input/output of a database determining an infusion amount, i.e., an example of storage database material.

FIG. 3 is a flowchart illustrating a drug infusion control method according to an exemplary embodiment of the present invention.

### BEST MODE

The present invention also provides a drug infusion control method including calculating and storing a drug infusion amount using weight data of experimental animals according to a preset formula, loading data on the drug infusion amount data stored, determining an operation time of an opening/closing valve, receiving an operation signal from an infusion switch, opening the opening/closing valve for a predetermined time, and repeatedly infusing a drug in response to the loaded data.

The drug infusion control method may further include capturing, after the opening of the opening/closing valve, an image of an airway of the animal after the drug is infused and outputting the captured image to a display unit and simultaneously storing the captured image in an image storage database.

Exemplary embodiments of the present invention will hereinafter be described in detail with reference to the accompanying drawings.

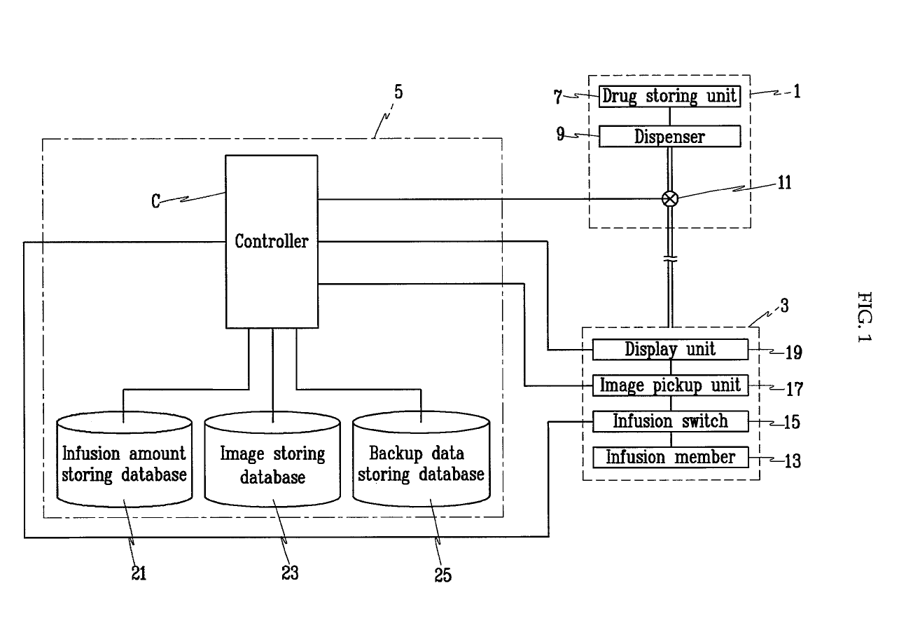

FIG. 1 is a schematic view of a drug infusion control apparatus according to an exemplary embodiment of the present invention. The drug infusion control apparatus includes a drug supplying unit 1, a drug infusion unit 3, and an infusion amount control unit 5. The drug supplying unit 1 includes a drug storage unit 7 for storing a drug that will be infused into experimental animals, a dispenser 9 for dispensing the drug from the drug storage unit 7 to the drug infusion unit, and an opening/closing valve 11 for adjusting an amount of the drug dispensed from the dispenser 9. The drug storage unit 7 may be a well-know drug storage unit.

The dispenser 9 for dispensing the drug stored in the drug storage unit 7 to the drug infusion unit 3 functions as a pump. Any device that can dispense the drug to the drug infusion unit may be used as the dispenser 9. An opening/closing time of the opening/closing valve 11 is controlled by a controller to adjust the amount of the drug dispensed to the drug infusion unit by the dispenser 9. The opening/closing valve 11 may be a conventional valve.

The drug infusion unit 3 includes an infusion member 13 for infusing the drug from the dispenser 9 into an oral cavity or airway of the animals. The drug infusion unit 3 further includes an infusion switch 15, an image pickup unit 17 for capturing an image of the oral infusion location or an inhalation infusion location, and a display unit 19 for displaying the captured image.

The infusion switch 15 is connected to a controller C, which will be described later, to send an operation signal to the controller C. The image pickup unit 17 may be a conventional camera that is coupled to the infusion member 13 to capture an image of a drug infusion location when the drug is infused into the animals. The image captured by the image pickup unit 17 is output through the display unit 19.

The infusion member 13, the infusion switch 15, the image pickup unit 17, and the display unit 19 are combined in the form of a single assembly so that a user can easily handle the same. The drug supplying unit 1 and the drug infusion unit 3 are not limited to specific structures. Well-known conventional units may be used as the drug supplying unit 1 and the drug infusion unit 3.

The infusion amount controller 5 includes the controller C, an infusion amount storage database 21, an image storage database 23, and a backup data storage database 25.

The controller C can control the closing/opening time of the opening/closing valve 11 in response to an input signal from the infusion switch 15. The controller C includes a process module formed with a program that can load weight values of the experimental animals, calculate an amount of the drug required for the experiments according to a preset formula, and store the calculated infusion amount data in the infusion amount storage database 21.

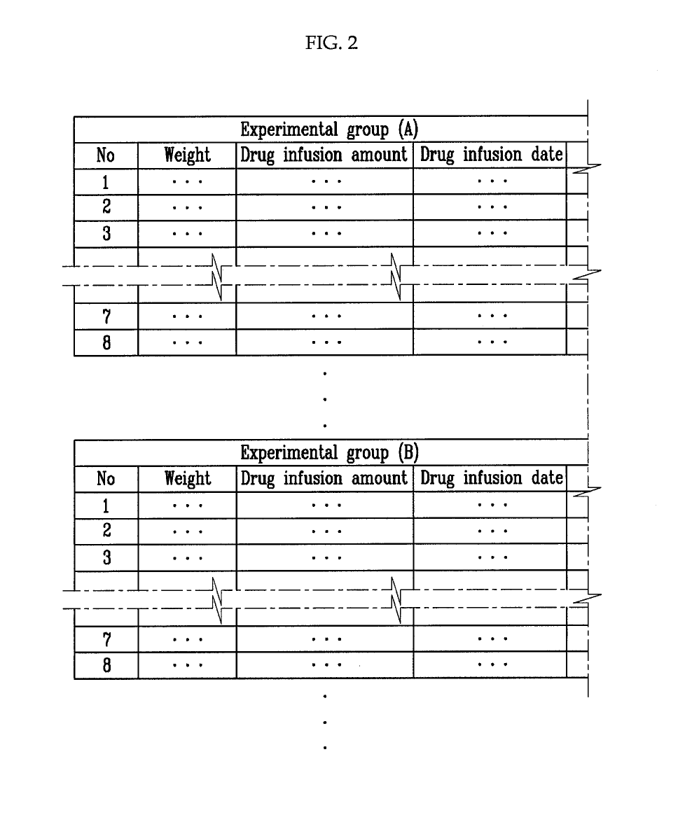

The infusion amount storage database 21 may store the number of animals in each experimental group and the weight of the experimental animals in each experimental group (see FIG. 2). The data stored in the infusion amount storage database 21 is not specifically limited. For example, the infusion amount storage database 21 may further store a drug infusion date, a drug infusion time, an amount of the drug infused to the animals, a kind of drug, and data required for performing the experiments on the animals before performing a clinical demonstration. Needless to say, other data except for data related to the drug infusion amount may be stored in a separate database. The image storage database 23 stores an image captured by the image input unit 17 of the drug infusion unit 3. That is, the drug is infused into the animals through oral cavities or airways of the animals during the experiments. The image pickup unit 17 captures an image of the drug infusion states so that a researcher can identify if the drug is normally infused and if there is any problem after the drug is infused. The image data are obtained whenever the drug is infused into the experimental animals and stored in the image storage database 23. After the drug is infused into the animals, the image data can be easily loaded so that the researcher can identify if the drug is normally infused.

The backup data storage database 25 is used to backup data stored in the infusion amount storage database 21 and the image storage database 23.

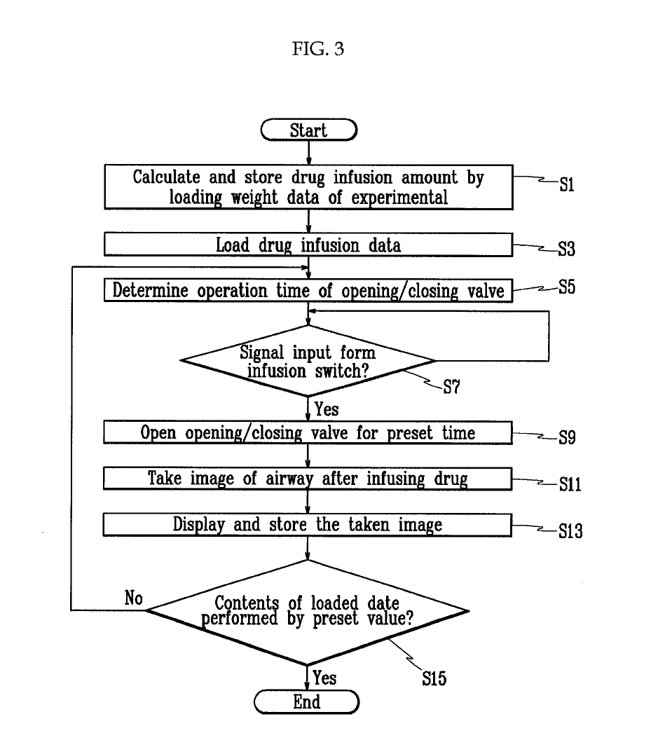

The following will describe a drug infusion control method using the above-described drug infusion control apparatus with reference to FIG. 3. The controller C of the infusion amount controller 5 loads weight data of the experimental animals, which are preset in the infusion amount database 21, calculates a drug infusion amount using the loaded data in accordance with a preset formula, and stores the calculated drug infusion amount in the infusion amount storage database 21 (S1). The above-described process is repeatedly performed for a predetermined period for animals in low and high dose groups. At this point, the infusion amount is automatically calculated in accordance with fluctuation of weights of the animals, and the calculated amount of the drug is infused to the animals. Therefore, the reliability of the experiment results can be improved. Needless to say, the weight of the animals may be measured and recorded by the researcher. Alternatively, the weight of the animals may be automatically measured and recorded in the infusion amount storage database.

The controller C sequentially selects the experimental animals from each of the experimental groups in numerical order and loads the drug infusion amount data (S3). The controller C calculates and determines an operation time of the opening/closing valve 11 based on the drug infusion amount data (S5). The controller C awaits an operation signal input from the infusion switch 15 (S7). When the operation signal is input from the infusion switch 15, the controller C opens the opening/closing valve 11 for a predetermined time (S9). After opening the opening/closing valve 11, the controller C controls the image pickup unit 17 to capture an image of an infusion location (S11). The controller C outputs the image captured by the image pickup unit to the display unit 19 and stores the image in the image storage database 23 (S13). The controller C determines if all the processes are sequentially performed (S15). If the processes are not completely performed, the above-described processes are repeated and the process ends. According to the drug infusion control apparatus and method, an infusion amount of the drug is automatically calculated in accordance with the weight of the experimental animals and thus the experiments can be quickly performed. In addition, the data control process is automatically performed, so experiments requiring more precise experimental conditions can be easily performed.

What is calimed is:

1. A drug infusion control apparatus comprising:
a drug supplying unit comprising an opening/closing valve;
a drug infusion unit for infusing a drug supplied from the drug supplying unit into experimental animals; and
an infusion amount controller for controlling the opening/closing valve of the drug supplying unit in accordance with a signal from an infusion switch of the drug infusion unit,
wherein the infusion amount controller comprises
an infusion amount storage database storing data on weights of the experimental animals and an infusion amount, and
a controller that controls the opening/closing valve to infuse the drug infusion amount calculated based on the weight data of the experimental animals stored in the infusion amount storage database into the experimental animals in accordance with a signal from the infusion switch.

2. The drug infusion control apparatus of claim 1, wherein the drug supplying unit further comprises:
a drug storing unit for storing the drug that will be infused into the experimental animals; and
a dispenser for dispensing the drug stored in the drug storing unit

3. The drug infusion control apparatus of claim 1, wherein the drug supplying unit further comprises:
a drug storing unit for storing the drug that will be infused into the experimental animals; and
a pump for supplying the drug stored in the drug storing unit.

4. The drug infusion control apparatus of claim 1, wherein the drug infusion unit further comprises:
an infusion member for infusing the drug from the drug supplying unit into the experimental animals;
an image pickup unit for capturing an image of an infusion state of the drug; and
a display unit for outputting the image that is captured by the image pickup unit to a display unit

5. The drug infusion control apparatus of claim 1, wherein the infusion amount controller further comprises an image storage database for storing the image that is captured by the image pickup unit of the drug infusion unit.

6. The drug infusion control apparatus of claim 1, wherein the infusion amount controller further comprises a backup storage database for storing the infusion amount data and the image data.

7. A drug infusion control method comprising:
calculating and storing a drug infusion amount using weight data of experimental animals according to a preset formula;
loading data on the drug infusion amount data that is stored;
determining an operation time of an opening/closing valve;
receiving an operation signal from an infusion switch;
opening the opening/closing valve for a predetermined time; and
repeatedly infusing a drug in response to the loaded data by a preset value after the opening of the opening/closing valve.

8. The drug infusion control method of claim 7, further comprising:
capturing, after the opening of the opening/closing valve, an image of an airway of the animal after the drug is infused; and
outputting the captured image to a display unit and simultaneously storing the captured image in an image storage database.
